(19) United States
(12) Patent Application Publication
(10) Pub. No.: US 2010/0178326 A1
(43) Pub. Date: Jul. 15, 2010

(54) AMINOPHOSPHONATE CARRIERS FOR LIPOSOME-BASED DRUG RELEASE SYSTEMS

(76) Inventors: Evgueni Pinkhassik, Memphis, TN (US); Delia C. Danila, Memphis, TN (US)

Correspondence Address: WYATT, TARRANT & COMBS, LLP, 1715 AARON BRENNER DRIVE, SUITE 800, MEMPHIS, TN, 38120-4367, US

(21) Appl. No.: 12/440,710

(22) PCT Filed: Mar. 10, 2009
(86) PCT No.: PCT/US09/36677

§ 371 Date: Mar. 10, 2009

Related U.S. Application Data
(60) Provisional application No. 61/035,332, filed on Mar. 10, 2008.

(51) Int. Cl. A61K 9/127

(52) U.S. Cl. 424/450

(57) ABSTRACT

A novel class of carriers for liposome-based drug delivery and release systems is provided. Such carriers exhibit excellent compatibility with hydrophilic drug compounds as well as effective release upon transport through phospholipid bilayers of cell membranes. Furthermore, such carriers are non-cytotoxic, particularly in terms of contact with cell membranes themselves. As such, these carriers accord an excellent manner of transporting pharmaceutical and other like compounds and materials to a user's individual cells for delayed release thereof at the site necessary to effectuate proper treatment. Drug delivery materials including such carriers are encompassed within this invention, as well as the overall method of drug delivery and delayed release provided thereby.

### CROSS-REFERENCE TO RELATED APPLICATIONS

This application claims benefit of U.S. Provisional Application Ser. No. 61/035,332 filed Mar. 10, 2008.

### STATEMENT REGARDING FEDERALLY SPONSORED RESEARCH OR DEVELOPMENT

The subject matter of this application has been funded through a grant, number CHE-0349315, from the National Science Foundation.

### FIELD OF THE INVENTION

A novel class of carriers for liposome-based drug delivery and release systems is provided. Such carriers exhibit excellent compatibility with hydrophilic drug compounds as well as effective release upon transport through phospholipid bilayers of cell membranes. Furthermore, such carriers are non-cytotoxic, particularly in terms of contact with cell membranes themselves. As such, these carriers accord an excellent manner of transporting pharmaceutical and other like compounds and materials to a user's individual cells for delayed release thereof at the site necessary to effectuate proper treatment. Drug delivery materials including such carriers are encompassed within this invention, as well as the overall method of drug delivery and delayed release provided thereby.

### BACKGROUND OF THE INVENTION AND PRIOR ART

A significant problem has arisen over the years concerning the administration of proper dosages of pharmaceuticals to targeted patients to ensure maximum efficacy of the medicine or medicines prescribed and, simultaneously, to minimize undesired side effects. Most pharmaceuticals are manufactured in a campaign form with a limited range of dosage strengths. For instance, many analgesics are provided by the manufacturer in two different dosage levels (such as 50 mg or 100 mg tablets). A physician or his patient thus has been forced to rely upon a number of different, less than reliable, manners of administering or prescribing a proper dose for maximum effectiveness. As a result, instances have occurred where patients have suffered toxic or adverse reactions due to an overdose of certain pharmaceuticals as well as many examples of ineffectiveness of certain drugs due to the inability of the target patient to absorb sufficient amounts of drugs for salutary treatment to occur.

Numerous techniques exist in the prior art for preparing sustained or controlled release pharmaceutical formulations in attempts to overcome this problem, including, without limitation, surrounding an osmotically active drug core with a semi permeable membrane. In such a manner, the pharmaceutical active becomes released from the drug core over time through exposure to gastrointestinal fluids which permeate the coating membrane and dissolve the active, thus permitting diffusion of the API through the membrane or orifice. Another non-limiting example is the encapsulation of a plurality of beads, pellets, or tablets that are coated with varying levels of diffusion barriers. Upon exposure to gastric fluids, the active may be released via a host of mechanisms such as diffusion, rupturing, eroding, and the like. Yet another manner of providing controlled release pharmaceuticals involves film coating, wherein one of a plurality of films requires drug diffusion through the film or dissolution of the film prior to API release within the body. Yet another manner of providing controlled release pharmaceuticals involves the formulation and compression of erodible or non-erodible, hydrophilic hydrogels or hydrophobic swelling or non-swelling matrices. Continuous treatments have been developed as well that dose pharmaceuticals within a user's body at a regular rate, although reliability of such treatments to perform such a highly regimented function has been suspect.

It has been known that dosages may be regulated through the utilization of different types of drug delivery systems. At the cellular level, particularly for continuous internal treatment drug delivery has been rather difficult to regulate, particularly in terms of dosage, time, and rate. In the past, organic molecules, including pharmaceutical materials, were transported into individual cells through rather elegant techniques, such as artificial ion channeling and supported liquid membrane movement. Although these procedures have shown promise, there are certain drawbacks, most importantly in terms of the difficulty in crossing a bilayer cell membrane without destroying the cell itself, the high selectivity such phospholipids exhibit in terms of allowing materials to pass through for any reason, not to mention the very quick transport rates such systems exhibit (the quicker the pass-through of a cell membrane, the potentially greater the tendency of the carrier to prove cytotoxic to the individual cell itself). Methods of meeting these base concerns were then developed and improved over time.

In particular, liposome-based systems, those that rely upon micellular structures for contact with phospholipid bilayers of individual cell membranes, generally exhibit acceptable delivery of properly hydrophilic pharmaceutical compounds through the protection of the drug from external exposure until contact with the target cell membranes within a user's body. In that manner, the liposome, being a bilayer structure itself and thus similar in structure to the targeted cell membrane, passes a protected drug from within its structure through to the cell membrane. To do so, however, a carrier is needed not only to properly transport the desired pharmaceutical compound out of the liposome and into the targeted cell, but also to perform such transport at the necessary time and at the correct rate.

Certain types of carriers have been developed for such purposes, including “needle and thread concept” carriers and photoresponsive carriers. In the “needle and thread” concept, a molecular umbrella binds covalently to an oligonucleotide chain and shields the hydrophilic region of two nucleotide units of the chain to help it penetrate a phospholipid bilayer (V. Janout, S. L. Regen, J. Am. Chem. Soc. 2005, V. 127, p. 22). Upon UV irradiation, a photoresponsive carrier undergoes a reversible transformation to a zwitterionic form that can bind amino acids through electrostatic interactions and facilitate transmembrane transport (J. Sunamoto et al. J. Am. Chem. Soc. 1982, V. 104, p. 5502). Unfortunately, these alternatives have not met all of the necessary requirements for a liposome-based drug delivery carrier. Specialized, reliable, safe techniques of such continuous internal drug delivery are therefore highly desired.

### SUMMARY AND ADVANTAGES OF THE INVENTION

Therefore, it is an advantage of this invention to provide a safe and reliable continuous internal pharmaceutical delivery system for individual patients. Another advantage of the invention is the ability to provide different carriers through selectivity of starting materials in carrier production in relation to the hydrophobic-hydrophilic structures of the target drug to be utilized. A further advantage of this invention is the ability to provide a safe, non-cytotoxic carrier compound that reliably delivers pharmaceutical doses without killing or otherwise damaging target cells.

Accordingly, this invention encompasses a liposome-based drug delivery system comprising a) a liposome; b) at least one drug component introduced within said liposome; and c) at least one aminophosphonate carrier introduced within said liposome. Such aminophosphonate carrier should be compatible with said at least one drug component such that when present within a user's body (i.e., a human or other mammal), said carrier functions to transport said drug component from within said liposome through a phospholipid bilayer for proper delivery to the targeted site of treatment. Thus, also encompassed within this invention is a method of treating a mammalian subject with at least one pharmaceutical compound, said method comprising the steps of: a) providing a liposome including at least one drug component and at least one compatible aminophosphonate carrier therein, b) ingesting said liposome of step “a”, c) allowing said carrier to transport the drug component out of said liposome and into the body of said mammalian subject. Further encompassed are materials including said liposome-based drug delivery systems, such as tablets, capsules, pills, beads, pellets, minitablets, powders, granules, suspensions, emulsions, and any combinations thereof.

### DETAILED DESCRIPTION OF THE INVENTION

For the purposes of this invention, the term “liposome” is intended to encompass any bilayer-type micellular structure. The term “drug delivery system” is intended to encompass a structure including components for complete delivery of pharmaceuticals from the structure and into a user's body (i.e., a liposome including a carrier and a drug).

This invention is based upon the ability to provide new and effective ways of delivering continuous, safe, and reliable dosages of pharmaceuticals from within a liposome into a user's body through the utilization of an acceptable, drug compatible carrier compound. It has been realized that aminophosphonate compounds (in particular α-aminophosphonates) exhibit all of these desired characteristics. Most notably, such specific types of compounds include a hydrogen bond donor moiety and a hydrogen bond acceptor moiety. Such a structure permits, at least, amino acids (including alanine, as one non-limiting example) to complex with the hydrophobic exterior of the liposome after transport therefrom. Furthermore, such compounds do not exhibit mutagenic properties, specifically at the cellular level, nor cytotoxicity. Perhaps most importantly, such compounds further exhibit excellent capabilities of transporting target pharmaceuticals through a liposome bilayer (and presumably through a cell membrane phospholipid bilayer as well) at a slow enough rate as to allow for excellent drug delivery and little to no lasting damaging effects on the targeted cell itself.

Aminophosphonates are produced through a Kabachnik-Fields condensation reaction between three starting materials: a primary amine, a carbonyl compound, and a phosphite compound. Such α-aminophosphonates thus include a hydrogen on the amine moiety, in order to provide a hydrogen bond donor group. Such starting materials may thus be of any type that meets the above broad description. Preferably, though not necessarily, however, for cost and steric reasons, the primary amine may be a C₂-C₁₈ amine or benzyl-based amine, the carbonyl compound may be an C₁-C₁₂ alkyl or alkenyl ketone (including cyclic groups thereon), and the phosphite may be C₁-C₂₄ alkyl or alkenyl as well.

The utilization of pharmaceutical treatments is based upon the ability to deliver needed drugs for treatment in a simple, reliable manner. It is thus the aim that such pharmaceutical utilization and delivery within a target patient provides the most effective results for treatment. The present invention provides a manner of reliable internal drug delivery for any type of pharmaceutical active and the formulation of a liposome with a drug and carrier provided in a form easily ingested by a user for such pharmaceutical purposes. Thus, the liposome-based drug delivery system of this invention may be in capsule and/or tablet form and may further include a plurality of different beads, pellets, powders, granules, emulsions, suspensions, and/or minitablets exhibiting different types and levels of coatings thereon and/or inert materials therein to permit tailored dissolution in intended body fluids, thereby permitting release of certain amounts of needed pharmaceutical actives to be absorbed at any desired rate into the body and within the correct region of the gastrointestinal tract for maximum effectiveness of treatment within the target patient's body. Coupled with the distinguishing carrier capabilities noted above, it is possible to deliver pharmaceutical in effective manners through such an overall system.

The active drug components which can be used according to the invention may be selected without limitation among those belonging to the following groups: amino acids, including alanine, phenylalanine, and the like; analgesic drugs such as, e.g., buprenorphine, codeine, fentanyl, morphine, hydromorphone, and the like; anti-inflammatory drugs such as, e.g., ibuprofen, indomethacin, naproxen, diclofenac, tolfenamic acid, piroxicam, and the like; anthelmintics such as albendazole, flubendazole, ivermectin, diethylcarbamazine citrate and the like. Antibacterials such as aminoglycosides (Kanamycin, Neomycin, and the like), Rifampin, cephalosporins and related beta lactams (Cefazolin, Cefuroxime, Cefaclor and the like), glycopeptides (Vancomycin and the like), penicillins (amoxicillin, ampicillin, carbenecillin, cloxacillin, dicloxacillin, and the like), quinolones (gatifloxcin, ciprofloxacin and the like), sulfonamides (sulfadiazine, sulfamethoxazole, sulfamerazine, trimethoprim, sulfanilamide, and the like), tranquilizers such as, e.g., diazepam, droperiodol, fluspirilene, haloperidol, lorazepam, and the like; cardiac glycosides such as, e.g., digoxin, ouabain, and the like; antiparkinson agents such as, e.g., bromocriptine, piperidin, benzhexol, benztropine, and the like; antidepressants such as, e.g., imipramine, nortriptyline, pritiptylene, lithium carbonate, clozapine, citalopram, fluoxeitine and the like; antineoplastic agents and immunosuppressants such as, e.g., cyclosporin A, fluorouracil, mercaptopurine, methotrexate, mitomycin, and the like; antiviral agents such as, e.g., idoxuridine, acyclovir, vidarabin, and the like; antibiotic agents such as, e.g., clindamycin, erythromycin, fusidic acid, gentamicin, and the like; antifungal agents such as, e.g., miconazole, ketoconazole, clotrimazole, amphotericin B, nystatin, and the like; antimicrobial agents such as, e.g., metronidazole, tetracyclines, and the like; appetite suppressants such as, e.g., fenfluramine, mazindol, phentermin, and the like; antiemetics such as, e.g., metoclopramide, droperidol, haloperidol, promethazine, and the like; antihistamines such as, e.g., chlorpheniramine, chlorpheniramine maleate, terfenadine, triprolidine, and the like; antimigraine agents such as, e.g., dihydroergotamine, ergotamine, pizotyline, and the like; coronary, cerebral or peripheral vasodilators such as, e.g., nifedipine, diltiazem, and the like; antianginals such as, e.g., glyceryl nitrate, isosorbide dinitrate, molsidomine, verapamil, and the like; calcium channel blockers such as, e.g., verapamil, nifedipine, diltiazem, nicardipine, and the like; hormonal agents such as, e.g., estradiol, estron, estriol, polyestradiol, polyestriol, dienestrol, diethylstilbestrol, progesterone, dihyroergosterone, cyproterone, danazol, testosterone, and the like; contraceptive agents such as, e.g., ethinyl estradiol, lynestrenol, etynodiol, norethisterone, mestranol, norgestrel, levonorgestrel, desogestrel, edroxyprogesterone, and the like; antithrombotic agents such as, e.g., warfarin, and the like; diuretics such as, e.g., hydrochlorothiazide, flunarizine, minoxidil, and the like; antihypertensive agents such as, e.g., propanolol, metoprolol such as metoprolol tartrate or metoprolol succinate, clonidine, pindolol, and the like; chemical dependency drugs such as, e.g., nicotine, methadone, and the like; local anesthetics such as, e.g., prilocaine, benzocaine, and the like; corticosteroids such as, e.g., beclomethasone, betamethasone, clobetasol, desonide, desoxymethasone, dexamethasone, diflucortolone, flumethasone, fluocinolone acetonide, fluocinonide, hydrocortisone, ethylprednisolone, triamcinolone acetonide, budesonide, halcinonide, and the like; dermatological agents such as, e.g., nitrofurantoin, dithranol, clioquinol, hydroxyquinoline, isotretionin, methoxsalen, methotrexate, tretionin, trioxsalen, salicylic acid, penicillamine, and the like; vitamins and the like; steroids such as, e.g., estradiol, progesterone, norethindrone, levonorgestrol, ethynodiol, levenorgestrel, norgestimate, gestanin, desogestrel, 3-keton-desogestrel, demegestone, promethoestrol, testosterone, spironolactone, and esters thereof, azole derivatives such as, e.g., imidazoles and mazoles and derivatives thereof, nitro compounds such as, e.g., amyl nitrates, nitroglycerine and isosorbide nitrates, amine compounds such as, e.g., pilocaine, oxyabutyninchloride, benzocaine, nicotine, chlorpheniramine, terfenadine, triprolidine, propanolol, metoprolol and salts thereof, oxicam derivatives such as, e.g., piroxicam, mucopolysaccharides such as, e.g., thiomucasee, opoid compounds such as, e.g., morphine and morphine-like drugs such as buprenorphine, oxymorphone, hydromorphone, levorphanol, hydrocodone, hydrocodone bitratrate, fentanyl and fentany derivatives and analogues, prostaglandins such as, e.g., a member of the PGA, PGB, PGE, or PGF series such as, e.g., misoprostol or enaprostil, a benzamide such as, e.g., metoclopramide, scopolamine, a peptide such as calcitonin, serratiopeptidase, superoxide dismutase (SOD), tryrotropin releasing hormone (TRH), growth hormone releasing hormone (GHRH), and the like, a xanthine such as, e.g., caffeine, theophylline, a catecholamine such as, e.g., ephedrine, salbutamol, terbutaline, a dihydropyridine such as, e.g., nifedipine, a thiazide such as, e.g., hydrochlorotiazide, flunarizine, a sydnonimine such as, e.g., molsidomine, and a sulfated polysaccharide, as well as cholesterol-lowering statin drugs, such as atorvastatin, simvastatin, and the like.

The active substances mentioned above are also listed for illustrative purposes; the invention is applicable to any pharmaceutical formulation regardless of the active substance or substances incorporated therein.

The concentration of the drug component (the dose) within the capsule, tablet, or other delivery system will depend primarily upon the desired degree of treatment sought for the target user. Furthermore, the carrier concentration within each liposome is in relation to the same basic purpose, albeit in relation to the amount of drug component(s) within the liposome and the desired rate of drug delivery from within the liposome itself. In general, an amount of carrier in a range of concentrations of 0.001-1.0% by weight of the liposome and a drug component in an range of amounts of 0.001-10% by weight of the liposome are potentially, though not necessarily, preferred.

If the liposome-based drug delivery system of this invention is included within a tablet or capsule, particularly including coated beads or pellets or minitablets thereof, such coatings may be of any standard material. Thus, coatings such as, e.g., a film coating, a sugar coating, a bioadhesive coating, or a so-called modified release coating, may be utilized for a number of reasons, including initial delayed delivery of the liposomes to the preferred region of a user's body prior to carrier transport commencement of the enclosed drug component. The coating provides a mechanism of obtaining the desired release profile of the liposome (and, ultimately, the drug component) included in the cores or, alternatively, masks the taste of bad-tasting active substances. In some cases, the cores according to the invention may contain two or more layers of coating e.g. a first coating which governs the release rate of the active substance and a second layer which is bioadhesive. Other combinations of coatings, including multiple coating configurations, are also within the scope of the present invention.

As mentioned above, the coating may provide the desired properties with respect to release of the liposome, as well as possible taste-masking. A suitable coating for a formulation according to the invention may, for example be a film coating, e.g. a coating based on one or more of the material selected from the following: hydroxypropylmethylcellulose, ethylcellulose, methylcellulose, hydroxyethylmethylcellulose, hydroxypropylcellulose, carboxymethylcellulose sodium, acrylate polymers (such as, e.g. EUDRAGIT® E, from Rohm Pharma), polyethylene glycols and polyvinylpyrrolidone; a sugar coating; a bioadhesive coating, such as, e.g., a coating comprising a bioadhesive substance such as, e.g. a fatty acid ester such as, e.g., fatty acid esters wherein the fatty acid component of the fatty acid ester is a saturated or unsaturated fatty acid having a total number of carbon atoms of from C₈ to C₂₂; specific examples are glyceryl monooleate, glyceryl monolinoleate, glycerol monolinolenate, or mixtures thereof. Also possible is a modified release coating, such as, e.g., an enteric coating, e.g. a coating which is such that when the coated materials are swallowed, it will be protected from the chemical, enzymatic and other conditions prevailing within the stomach during passage through this part of the digestive system, but will dissolve or otherwise disintegrate within the intestinal tract, thereby releasing the active substance within the intestines. An enteric coating may be based on one or more of the material selected from the following: methacrylic acid copolymers (e.g. EUDRAGIT® L or S), cellulose acetate phthalate, ethylcellulose, hydroxypropylmethylcellulose acetate succinate, polyvinyl acetate phthalate, and shellac; waxes such as, e.g., beeswax, glycowax, castor wax, carnauba wax; hydrogenated oils such as, e.g., hydrogenated castor oil, hydrogenated coconut oil, hydrogenated rape seed oil, hydrogenated soybean oil; fatty acid or fatty alcohol derivatives such as, e.g., stearyl alcohol, glyceryl monostearate, glyceryl distearate, glycerol palmitostearate; acrylic polymers such as, e.g., acrylic resins (EUDRAGIT® RL and RS acrylic resins are copolymers of acrylic and methacrylic acid esters with a low content of quaternary ammonium groups) poly(methyl methacrylate), methacrylate hydrogels, ethylene glycol methacrylate; polylactide derivatives such as, e.g., dl-polylactic acid, polylactic-glycolic acid copolymer; cellulose derivatives, such as, e.g., ethylcellulose, cellulose acetate, cellulose propionate, cellulose butyrate, cellulose valerate, cellulose acetate propionate, cellulose acetate butyrate; vinyl polymers such as, e.g., polyvinyl acetate, polyvinyl formal, polyvinyl butyryl, vinyl chloride-vinyl acetate copolymer, ethylene-vinyl acetate copolymer, vinyl chloride-propylene-vinyl acetate copolymer, polyvinylpyrrolidone; glycols such as, e.g., 1,3-butylene glycol, polyethylene glycols; polyethylene; polyester; polybutadiene; and other high molecular synthetic polymers. The coating material may be admixed with various excipients such as, e.g., plasticizers; anti-adhesives such as, e.g., silicon dioxide (silica), talc, and magnesium stearate, kaolin; colourants; and solvents in a manner known per se.

Examples of plasticizers for use in accordance with the invention include polyhydric alcohols such as, e.g., propylene glycol, glycerol, and polyethylene glycol; acetate esters such as, e.g., glyceryl triacetate (Triacetin), triethyl acetate, and acetyl triethyl acetate, triethyl citrate; phthalate esters such as, e.g., diethylphthalate; glycerides such as, e.g., acetylated monoglycerides; oils such as, e.g., castor oil, mineral oil, and fractionated coconut oil; and dibutyl sebacate.

In one potential non-limiting embodiment, the coating is applied on the pellets, beads, and/or minitablets from a solution and/or suspension in a non-toxic or low-toxicity organic solvent or in an aqueous medium. The coating may also be applied by electrostatic deposition. Utilization of an aqueous medium is preferred due to safety, economy and environment. The application of the coating, via aqueous and/or organic solvent application, may be performed in a fluidized bed but any suitable coating apparatus may be employed such as those well known by a person skilled in the art (e.g. pan coating, spray-drying, electrostatic coating etc.). When the cores are coated in a fluidized bed apparatus it has proved advantageous to apply the coating composition from a nozzle positioned in the bottom of the fluid bed apparatus, i.e. having the flow of the liquid (the coating composition) and the fluidizing air in a mixed flow except when the coating is performed with a fat or a wax. By using a mixed flow it has been shown that it is possible to coat relatively small particles without agglomeration.

The amount of coating applied on the pellets, beads, and/or minitablets depends, inter alia, on the size of the cores (such as granules, beads or minitablets), the type of coating employed, the amount and type of the active contained in the minitablets and/or beads, and the desired release pattern. In one potentially preferred, but non-limiting, embodiment, a core size of from about 500 to 1400 microns, more preferably from about 600 to about 1200 microns, is utilized with a coating of 0.1-15% weight gain employed in order to produce thin-coated beads; whereas the same size core (and thus the same amount of active) is supplied, albeit with larger amounts of coatings (such as one set of beads of about 15-25% weight gain, and a second set of even greater coating amounts, such as from 25-50% weight gain) in order to provide beads that, taken in combination with the first set, exhibit differing dissolution rates.

Other non-limiting embodiments of the liposome-based drug delivery systems could include minitablets, wherein the liposome is either coated on the tablet surface or compacted with a certain amount of inert materials that delay dissolution. Thus, varied formulations of minitablets comprising 1-99 parts of drug mixed with 99-1 parts of appropriate rate controlling excipient included within the drug delivery system will effectuate an analogous result to the coated beads and/or pellets note above. Such excipients can include, without limitation, rate-controlling water-swellable or water-erodible polymers that will react in the gastrointestinal tract to form a gel layer on the minitablet surface through which the liposome will diffuse/erode over time or will erode over time upon exposure to gastro-intestinal fluids to permit liposome release. Certain types of such polymers include, again without limitation, hydrocolloids, pectins, alginates, polyacrylamides (and homologues), polyacrylic acids (and homologues), polyethylene glycol, poly(ethylene oxide), polyvinyl alcohol, polyvinylpyrrolidones, starch (and like sugar-based molecules), modified starch, animal-derived gelatin, cellulose ethers (such as carboxymethylcellulose, hydroxyethylcellulose, and the like), and gums, such as carrageenan, guar, agar, arabic, ghatti, karaya, tragacanth, tamarind, locust bean, xanthan, and the like. The amount of excipient present in relation to the liposome level will determine the rate of liposome release/diffusion/erosion over time.

Other possible non-limiting embodiments of the liposome-based drug delivery system are micronized powders produced through but not limited to jet milling and/or powder mixtures produced by methods such as co-grinding via ball milling to facilitate intimate contact between the powders. Introducing differing mixtures of such powdered forms can thus be provided to dissolve in a manner analogous to the coated/uncoated beads and/or pellets noted above as well.

The drug/carrier-containing liposomes may also be delivered in the form of granules produced by wet, dry, and/or fluid bed granulation techniques. Modifications of particle aggregates can thus be utilized to provide differing dissolution rates for delayed delivery.

Yet another possible non-limiting embodiment for liposome delivery is suspending and/or dispersing such powder and/or powder mixtures through ball or colloid milling. Varying the suspending agent viscosity and/or flocculation mechanism can modify the drug release profile as needed. Possible suspending agents include, without limitation, water-soluble polymers, such as certain classes of alkylcelluloses and alkylalkylcelluloses, polyhydric alcohols (such as alkylene polyols and polyalkylene polyols), EO-PO copolymers or block copolymers, and any mixtures or combinations thereof.

Still another potential embodiment of the liposomes includes, again, without limitation, emulsions, such as single, micro-, and multiple emulsions. Combinations of immiscible liquids such as oil and water are admixed with surfactants to form emulsions. The drug may then be dissolved in one of the liquid phases and mixed with the remaining components to form active-containing droplets suspended in solution. Micro emulsions are formulated in the same manner as regular emulsions but yield micelles containing the drug-rich phase and appear transparent to the human eye. Examples of suitable emulsifying agents for this purpose include, without limitation, non-toxic food-grade surfactants, such as alkoxylated alcohols, sulfonated hydrocarbons, silicone-based surface-active agents, and the like.

The drug/carrier-containing liposomes contained in the capsule, tablet, or other delivery system may either be present in admixture with a pharmaceutically acceptable inert carrier, or it may be applied on inert cores comprising a pharmaceutically acceptable inert carrier, optionally in admixture with one or more pharmaceutically acceptable excipients (see below). In the latter case, the active substance may be applied by means of methods well known to a person skilled in the art such as, as one non-limiting example, a fluidized bed method. In the prepared materials, the liposomes are present in a layer on the outer surface of the uncoated carrier.

Apart from the liposomes and the pharmaceutically acceptable inert carrier, the pharmaceutical formulations according to the invention may contain other acceptable pharmaceutical-grade excipients. The pharmaceutically acceptable excipient for use in a particulate formulation according to the invention is generally selected from the group consisting of fillers, binders, disintegrants, glidants, and lubricants; in the following is given a more detailed list of suitable pharmaceutically acceptable excipients for use in formulations according to the invention. The choice of pharmaceutically acceptable excipient(s) in a formulation according to the invention and the optimum concentration thereof cannot generally be predicted and must be determined on the basis of an experimental evaluation of the final formulation. The formulation contains the active substance and the inert carrier in admixture with one or more pharmaceutical grade excipients. These excipients may be, for example, inert diluents or fillers, such as sucrose, sorbitol, sugar, mannitol, microcrystalline cellulose, starches including potato starch, cornstarch, tapioca, rice, and the like, calcium carbonate, sodium chloride, lactose, calcium phosphate, calcium sulfate, or sodium phosphate; granulating and disintegrating agents, for example, cellulose derivatives including sodium carboxymethylcellulose, croscarmellose, starches including sodium starch glycolate, potato starch, cross-linked polyvinylpyrrolidone (such as crospovidone), alginates, or alginic acid; binding agents, for example, sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone such as, e.g, PVP K12, PVP K15, PVP K17, PVP K25, PVP K30, PVP K60, PVP K90, or PVP K120, or combinations thereof, polyvinylacetate, or polyethylene glycol; and lubricating agents including glidants and antiadhesives, for example, magnesium stearate, zinc stearate, stearic acid, silicas, hydrogenated vegetable oils, or talc. Other pharmaceutically acceptable excipients can be colorants, flavoring agents, plasticizers, humectants, buffering agents, etc.

The general amounts of the coating components can be of any level to permit proper dissolution of the initial tablet and/or capsule (containing the liposome-based drug delivery systems) within a target patient's gastrointestinal tract. Likewise, any amount of additives, such as excipients, binders, disintegrating agents, etc., as noted above, may be of any acceptable level, usually from about 0.01 to about 99% by weight of the entire coating and/or minitablet formulation. The amount of active drug present may also be varied within the cores of different coated beads and/or different minitablet formulations present within a single delivery source, if necessary. There is thus no requirement that each bead and/or minitablet utilized within the delivery source (as one non-limiting example, a capsule) remain static for drug content or amount and type of coating applied or for amount and type of matrix polymer content.

### PREFERRED EMBODIMENTS OF THE INVENTION

The invention is hereinafter more particularly described through the following non-limiting examples. It is noted that specific pharmaceutical actives are utilized within these examples; however, it should be well understood by the ordinarily skilled artisan within the pertinent art that the inventive method may be practiced with any known active. Thus, the specific types listed below are in no way intended to indicate a limitation as to the breadth of this invention.

### Aminophosphonate Carrier Production

As noted above, aminophosphonate compounds can be produced via a one-pot synthesis Kabachnik-Fields reactions between a primary amine, a phosphite, and a carbonyl compound. Such a reaction is the condensation between these three individual compounds into the single aminophosphonate compound exhibiting a hydrogen bond donor moiety and a hydrogen bond acceptor moiety simultaneously. The following table lists the reactants that formed the tested aminophosphonate compounds for efflux and fluorescence experimentation thereafter.

TABLE 1
Aminophosphonate Examples

| | Reactants | | |
|---|---|---|---|
| Ex. # | Primary Amine | Carbonyl Compound | Phosphite Compound |
| 1 | benzylamine | acetone | dimethylphosphite |
| 2 | benzylamine | acetone | dibutylphosphite |
| 3 | benzylamine | acetone | bis-2-ethylhexyl- |
| | | | phosphite |
| 4 | benzylamine | cyclohexanone | dimethylphosphite |
| 5 | benzylamine | cyclohexanone | dibutylphosphite |
| 6 | aniline | acetone | dimethylphosphite |
| 7 | aniline | cyclohexanone | dibutylphosphite |

Such aminophosphonate compounds exhibited varied molecular weights and actual compound sizes, leading to different hydrophobic-hydrophilic balances for such compounds as well. The end products were isolated by column chromatography on silica gel using hexane/ethylacetate (5:1) as eluent. These compounds were then analyzed for carrier transport properties.

### Aminophosphonate Carrier Analyses

Two sets of tests were undertaken to determine the viability of such aminophosphonate compounds as drug carriers in liposome-based delivery systems. The first evaluated liposome efflux coupled with enzymatic assays. In such an experiment, unilamellar liposomes loaded with 300 mM of alanine (an amino acid) were prepared from dipalmitoyl phosphatidyl choline (DPPC) and cholesterol (3:1 molar ratio) (in accordance with Torchilin, V. et al., Liposomes 2003, Second Edition, Oxford Press). Several samples of alanine-loaded liposomes were then loaded further with identical amounts of the different aminophosphonate carriers from above (with DMSO as a solvent) thereby producing different samples with different carriers but with same concentrations. The liposomes were then measured for alanine efflux after one hour of exposure as the conversion of alanine to pyruvate, catalyzed by Glutamic-Pyruvic Transmaminase enzyme, and further to lactate, catalyzed by the enzyme Lactate Dehydrogenase, accompanied by the oxidation of nicotinamide cofactor NADH to NAD⁺ (as monitored by UV spectroscopy). The results are tabulated below (with a control of no carrier):

TABLE 2
Alanine Flux Measurements

| Carrier Example # | Alanine Flux |
|---|---|
| (from Table 1, above) | (nmol/h × m²) |
| Control | 0.7 |
| 1 | 4.4 |
| 2 | 3.1 |
| 3 | 4.3 |
| 4 | 7.2 |
| 5 | 4.4 |
| 6 | 7.8 |
| 7 | 4.4 |

These carriers thus exhibited acceptable, if not excellent, alanine flux measurements indicating proper liposomal release due to carrier transport through the bilayer structure.

Further testing was then undertaken to determine if such aminophosphonate carriers exhibited cell lysis or other cytotoxic possibilities. To do so, calcein, a self-quenching fluorescent dye, was introduced within the same liposomes as noted above. Above 100 mM concentrations, calcein loses 98% of its fluorescent capability. Such liposomes were mixed with aminophosphonate solutions in DMSO and the fluorescence thereof was then monitored for one hour (as for the alanine efflux above). No detectable change in fluorescence was observed. Subsequently, the liposomes were treated with TRITON X-100® to lyse the liposomes; an immediate increase of emission, indicative of dye dilution below self-quenching concentration due to release of calcein, was then observed. It was determined that the aminophosphonates did not cause the bilayer membranes to become more permeable as a result of this analysis. Thus, these carriers exhibit not only excellent transport kinetics and rates, but do not exhibit any appreciable level of cytotoxicity. Hence, such carriers make suitable compounds for liposomal-based drug delivery systems.

While certain preferred and alternative embodiments of the invention have been set forth for purposes of disclosing the invention, modifications to the disclosed embodiments may occur to those who are skilled in the art. Accordingly, this specification is intended to cover all embodiments of the invention and modifications thereof which do not depart from the spirit and scope of the invention.

What is claimed is:

1. A liposome-based drug delivery system comprising a) a liposome; b) at least one drug component introduced within said liposome; and c) at least one aminophosphonate carrier introduced within said liposome.

2. The liposome-based drug delivery system of claim 1 wherein said aminophosphonate is an α-aminophosphonate.

3. The liposome-based drug delivery system of claim 1 wherein said delivery system is incorporated within a composition in a form selected from the group consisting of tablets, capsules, pills, beads, pellets, minitablets, powders, granules, suspensions, emulsions, and any combinations thereof.

4. The liposome-based drug delivery system of claim 2 wherein said delivery system is incorporated within a composition in a form selected from the group consisting of tablets, capsules, pills, beads, pellets, minitablets, powders, granules, suspensions, emulsions, and any combinations thereof.

5. A method of treating a mammalian subject with at least one pharmaceutical compound, said method comprising the steps of: a) providing a liposome including at least one drug component and at least one compatible aminophosphonate carrier therein, b) ingesting said liposome of step “a”, c) allowing said carrier to transport the drug component out of said liposome and into the body of said mammalian subject.

6. The method of claim 1 wherein said aminophosphonate is an α-aminophosphonate.

7. The method of claim 5 wherein said liposome is incorporated within a composition in a form selected from the group consisting of tablets, capsules, pills, beads, pellets, minitablets, powders, granules, suspensions, emulsions, and any combinations thereof.

8. The method of claim 6 wherein said liposome is incorporated within a composition in a form selected from the group consisting of tablets, capsules, pills, beads, pellets, minitablets, powders, granules, suspensions, emulsions, and any combinations thereof.
